(19) United States
(12) Patent Application Publication
(10) Pub. No.: US 2012/0189652 A1
(43) Pub. Date: Jul. 26, 2012

(54) FREE RADICAL-INDUCED INTRACELLULAR DNA DAMAGE REDUCING REAGENT, MEDICAMENT OR HEALTH FOOD AND SKIN CARE PRODUCT OR COSMETIC, AND METHODS FOR PREPARING THE SAME

(75) Inventors: Jing-Iong Yang, Kaohsiung (TW); Hsueh-Wei Chang, Kaohsiung (TW); Chao-Neng Tseng, Kaohsiung (TW); Jin-Ching Lee, Kaohsiung (TW); Chih-yuan Lin, Pingtung County (TW); Chi-Chen Yeh, Pingtung County (TW)

(73) Assignees: KAOHSIUNG MEDICAL UNIVERSITY, Kaohsiung (TW); NATIONAL KAOHSIUNG MARINE UNIVERSITY, Kaohsiung (TW)

(21) Appl. No.: 13/033,308

(22) Filed: Feb. 23, 2011

(30) Foreign Application Priority Data
Jan. 21, 2011 (TW) 100102240

(51) Int. Cl. A61K 36/04

(52) U.S. Cl. 424/195.17

(57) ABSTRACT

The invention provides a free radical-induced intracellular DNA damage reducing reagent, including: an effective amount of a Gracilaria sp. extract, serving as an active ingredient, wherein the free radical-induced intracellular DNA damage reducing reagent is capable of reducing free radical-induced intracellular DNA damage. The invention also provides a free radical-induced intracellular DNA damage reducing medicament or health food and a free radical-induced intracellular DNA damage reducing skin care product or cosmetic.

### CROSS REFERENCE TO RELATED APPLICATIONS

This application claims priority of Taiwan Patent Application No. 100102240, filed on Jan. 21, 2011, the entirety of which is incorporated by reference herein.

### BACKGROUND OF THE INVENTION

1. Field of the Invention

The present invention relates to a seaweed extract, and in particular relates to a Gracilaria sp. extract which has anti free radical effect and is capable of reducing free radical-induced intracellular DNA damage.

2. Description of the Related Art

Reactive oxygen species including superoxide anion radicals, hydroxyl radicals and hydrogen peroxide are produced as an inevitable result of cellular metabolism or by environmental factors (Carcinogenesis, 2009, 30, 2070-2076). Excessive free radicals are also found to inflict cellular injuries such as lipid oxidation, gene dysregulation, altered protein function, and DNA damage and mutation, as well as retarded cell growth. Therefore, free radical stress (or oxidative stress) contributes significantly to aging (J Clin Oncol, 2007, 25, 1844-1851) and cancer formation in humans (Lancet, 1994, 344, 862-863).

Previous studies have shown that seaweed derivatives have potential anti-cancer capabilities, which inhibit the growth of a number of human cancer cell lines including the promyelocytic leukemia cell line HL-60 (Biochemistry (Mosc), 2006, 71, 1312-1315), the colon adenocarcinoma cell line HT-29 (J Med Food, 2007, 10, 587-593) and the breast adenocarcinoma cell line MCF7 (J Agric Food Chem, 2009, 57, 8677-8682). Seaweed extracts have also been shown to have protective effects against hypercholesterolemia and free radical stress (Phytother Res, 2005, 19, 506-510) and promote the growth of probiotics (Pak J Pharm Sci, 1991, 4, 49-54).

The major seaweed species found in the surrounding areas of Taiwan include Gracilaria tenuistipitata, Gracilaria coforvoides, Gracilaria gigas, Gracilaria chorda and Gracilaria compressa. Seaweed extracts from some of these species have been reported to exert anti-aging activity (Journal of Plant Diseases and Protection, 2009, 116, 263-270) and contain rich sources of amino acids, fatty acids, vitamins, minerals, poly phenolic compounds and carbohydrates (Food Control, 2007, 18, 639-645). Gracilaria tenuistipitata is edible and can be used as an ingredient of foodstuffs and cosmetics. It is the major species of Gracilaria currently cultivated in Taiwan. A previous report has shown that Gracilaria tenuistipitata exhibits radical scavenging activities that are sensitive to autoclaving (Plant Foods Hum Nutr, 2009, 64, 218-223).

The common antioxidants found in seaweed extracts include phenolics, flavonoids, and ascorbic acid, which function as radical scavengers. Typical phenolic compounds that possess antioxidant activity are known to be mainly phenolic acids and flavonoids (J. Agric. Food Chem., 1999, 47, 3954-3962). It has been reported that algal polyphenols contribute to the main antioxidant activity of seaweeds or their extracts (Hydrobiology, 1996, 326/327, 199-203; J Appl Phycol 2008, 20, 705-711; Food Chemistry, 2009, 112, 575-581.). In fact, many previous studies have reported a high correlation between 1,1-diphenyl-2-picrylhydrazyl (DPPH) radical scavenging and the phenolic contents in plant foods, such as cereals (Journal of Cereal Science, 2001, 33, 97-103), herbs (J Agric Food Chem., 2001, 49(11), 5165-5170), fruits (J Sci Agric Food, 2000, 80:2021-2027; Journal of Agricultural and Food Chemistry, 2001, 49, 5489-5493), seaweeds (Journal of Agricultural and Food Chemistry, 2001, 49, 5489-5493), and vegetables (Food Chemistry 2004, 85(1), 19-26). Flavonoids are one of the most widespread and diverse phenolics and they are widely known antioxidants which can trap and neutralize free radicals, quench singlet and triplet oxygen, or decompose peroxides (Food Chemistry, 1999, 66(4), 401-436). In addition to phenolic compounds, the functional and bioactive activity of ascorbic acid present in plant foods has been reported (Public Health Nutr., 2004 7(3), 407-422). Ascorbic acid is an effective antioxidant and is well-known for its chelating action (Food Chemistry, 2008, 107, 40-43) and is richly available in most seaweeds (Hydrobiologia, 1987, 477-481; FAO Nutr Stud., 1974, 1-66).

### BRIEF SUMMARY OF THE INVENTION

The invention provides a free radical-induced intracellular DNA damage reducing reagent, comprising: an effective amount of a Gracilaria sp. extract, serving as an active ingredient, wherein the free radical-induced intracellular DNA damage reducing reagent is capable of reducing free radical-induced intracellular DNA damage.

The invention also provides a free radical-induced intracellular DNA damage reducing medicament or health food, comprising: an effective amount of a Gracilaria sp. extract, serving as an active ingredient, wherein the free radical-induced intracellular DNA damage reducing medicament or health food is capable of reducing free radical-induced intracellular DNA damage.

The invention also provides a free radical-induced intracellular DNA damage reducing skin care product or cosmetic, comprising: an effective amount of a Gracilaria sp. extract, serving as an active ingredient, wherein the free radical-induced intracellular DNA damage reducing skin care product or cosmetic is capable of reducing free radical-induced intracellular DNA damage.

The invention further provides a method for preparing a free radical-induced intracellular DNA damage reducing reagent, comprising: providing an effective amount of a Gracilaria sp. extract, as an active ingredient, in preparation of a free radical-induced intracellular DNA damage reducing reagent.

The invention further provides a method for preparing a free radical-induced intracellular DNA damage reducing medicament or health food, comprising: providing an effective amount of a Gracilaria sp. extract, as an active ingredient, in preparation of a free radical-induced intracellular DNA damage reducing medicament or health food.

The invention further provides a method for preparing a free radical-induced intracellular DNA damage reducing skin care product or cosmetic, comprising: providing an effective amount of a Gracilaria sp. extract, as an active ingredient, in preparation of a free radical-induced intracellular DNA damage reducing skin care product or cosmetic.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1A shows 1,1-diphenyl-2-picrylhydrazyl (DPPH) radical scavenging activity of the extract from Gracilaria at different concentrations (1-4 mg/ml). Data is represented as mean±SD (n=3). *P<0.001 (versus the extract from Gracilaria at 1 mg/ml);

FIG. 1B shows 1,1-diphenyl-2-picrylhydrazyl radical scavenging activity of the positive controls, BHA (10 ppm) and Vitamin C (100 ppm), and 4 mg/ml of the extract from Gracilaria. Data is represented as mean±SD (n=3). *P<0.001 (versus the control (the extract from Gracilaria));

FIG. 2 shows a protective effect of the extract from Gracilaria on H₂O₂ induced plasmid conformational change. FIG. 2A shows a gel electrophoresis view for H₂O₂ treated plasmid DNA in the presence of the extract from Gracilaria at different concentrations (1-4 mg/ml). FIG. 2B shows the supercoiled content for H₂O₂ treated plasmid DNA in the presence of the extract from Gracilaria at different concentrations (1-4 mg/ml). Data is represented as mean±SD (n=3). *1′<0.001 (versus H₂O₂ only);

FIG. 3 shows a protective effect of the extract from Gracilaria on H₂O₂ induced cellular DNA strand breaks in comet assay. FIG. 3A shows a photograph for comet-nuclear extract assay for the negative control (normal cell). FIG. 3B shows a photograph for comet-nuclear extract assay for the positive control (cells treated with H₂O₂ only). FIGS. 3C-3E show photographs for comet-nuclear extract assay for the cells treated with 1-4 mg/ml of the extract from Gracilaria in the presence of H₂O₂, respectively. FIG. 3F shows in comet-nuclear extract assay, % tail DNA for negative control, the positive control and the cells treated with 1-4 mg/ml of the extract from Gracilaria, respectively. Data represents in mean±SD (n=200). *P<0.001 (versus H₂O₂ only);

FIG. 4 shows cell viability. FIG. 4A shows cell viability for cells treated without H₂O₂ in the presence of the extract from Gracilaria (0, 0.5, 1, 2, and 4 mg/ml), for 24 hours. FIG. 4B shows cell viability for cells treated with H₂O₂ in the presence of the extract from Gracilaria (0, 0.5, 1, 2, and 4 mg/ml), for 24 hours. Data is represented as mean±SD (n=3). *P<0.001 (versus H₂O₂ only); and

FIG. 5 shows 24 hour cell cycle distribution for cells treated with or without H₂O₂ in the presence of the extract from Gracilaria (0, 0.5, 1, 2, and 4 mg/ml). Data is represented as mean±SD (n=3).

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In one aspect of the invention, the invention provides a Gracilaria sp. extract which is capable of scavenging free radicals.

The foregoing Gracilaria sp. may comprise Gracilaria tenuistipitata, Gracilaria coforvoides, Gracilaria gigas, Gracilaria chorda, Gracilaria lichenoides or Gracilaria compressa.

A process for obtaining the Gracilaria sp. extract of the invention may comprise performing a first extraction step to the Gracilaria sp. to obtain a first extract. The first extraction step may comprise extracting the Gracilaria sp. with a solvent.

The solvent used for extracting the Gracilaria sp. may comprise, and is not limited to, water, methanol, ethanol, propanol or the combination thereof. In one embodiment, the Gracilaria sp, used in the first extraction step is Gracilaria tenuistipitata, and the solvent used is water.

In one embodiment, in the first extraction step, a ratio of the Gracilaria sp to the solvent is about 1:15 (w/v) to 1:25 (w/v), preferably, 1:20 (w/v).

In one embodiment, a performing temperature for the first extraction step is about 15-35° C., preferably room temperature or about 24-26° C. Moreover, in one embodiment, a performing time for the first extraction step is about 15-30 hours, preferably about 24 hours.

In another embodiment, the process for obtaining the Gracilaria sp. extract may further comprise filtering the first extract to obtain a filtrate and a residue, wherein the filtrate is a second extract and used as the Gracilaria sp. extract of the invention.

Furthermore, in further another embodiment, the process for obtaining the Gracilaria sp. extract may further comprise the following steps. First, after filtering the first extract to obtain the filtrate and the residue, a second extraction step is performed to the residue to obtain an extract of the residue, wherein the second extraction step comprises extracting the residue with a second solvent. Next, the extract of the residue is filtered to obtain a second filtrate. Finally, the second filtrate is mixed with the second extract to obtain a third extract, and the third extract is used as the Gracilaria sp. extract of the invention.

The second solvent used in the second extraction step may comprise, and is not limited to, water, methanol, ethanol, propanol or the combination thereof. The solvent used in the first extraction step and the second solvent used in the second extraction step may be the same or different. In one embodiment, the second solvent used in the second extraction step is water. Moreover, a performing temperature for the second extraction step is about 15-35° C., preferably room temperature or about 24-26° C. Furthermore, in one embodiment, a performing time for the second extraction step is about 15-30 hours, preferably about 24 hours.

In further another embodiment, the Gracilaria sp. extract obtained as mentioned above may be further concentrated under reduced pressure and/or lyophilized.

In one embodiment, the content of the total phenolics, flavonoid and ascorbic acid of the per milligram (mg) dry Gracilaria sp. extract of the invention may be about 93-105 μg gallic acid equivalent, 17-27 μg quercetin equivalent and 0.1-6 μg, respectively. Preferably, the content of the total phenolics, flavonoid and ascorbic acid of the per milligram (mg) dry Gracilaria sp. extract of the invention may be about 95-101 μg gallic acid equivalent, 19-25 μg quercetin equivalent and 0.5-4 μg, respectively.

The Gracilaria sp. extract of the invention has a free radical scavenging effect. In one embodiment, free radicals which may be scavenged by the Gracilaria sp. extract may comprise hydroxyl radicals, superoxide anion radicals, 1,1-diphenyl-2-picrylhydrazyl (DPPH) radical, etc., but is not limited thereto. In one embodiment, the Gracilaria sp. extract has a scavenging effect on 1,1-diphenyl-2-picrylhydrazyl (DPPH) radical. In another embodiment, the Gracilaria sp. extract is capable of scavenging hydroxyl radicals generated from hydrogen peroxide.

In one embodiment, the Gracilaria sp. extract further has an effect for reducing free radical-induced DNA damage and/or has an effect for reducing cell death or cell cycle arrest under free radical or oxidative stress. The free radical-induced DNA damage may comprise free radical-induced DNA damage in a cell-free condition or free radical-induced intracellular DNA damage.

The free radical-induced DNA damage in the cell-free condition may comprise free radical-induced non cellular or extracellular DNA damage, such as plasmid DNA damage induced by free radicals outside of a cell, but is not limited thereto. The free radical-induced intracellular DNA damage may comprise intranuclear DNA damage induced by free radicals, such as genomic DNA damage induced by free radicals, or other DNA damage in a cell, but is not limited thereto.

Therefore, in another aspect of the invention, the invention may provide a free radical-induced intracellular DNA damage reducing reagent which is capable of reducing free radical-induced intracellular DNA damage, and the reagent may comprise an effective amount of the above-mentioned Gracilaria sp. extract, serving an active ingredient. In addition, the invention may further provide a method for preparing a free radical-induced intracellular DNA damage reducing reagent. The method comprises providing an effective amount of the above-mentioned Gracilaria sp. extract, as an active ingredient, in preparation of a free radical-induced intracellular DNA damage reducing reagent.

In one embodiment, the free radical-induced intracellular DNA damage reducing reagent mentioned above may further have an effect for reducing cell death or cell cycle arrest under free radical stress or oxidative stress.

In one embodiment, the free radical-induced intracellular DNA damage reducing reagent mentioned above may further comprise a pharmaceutically acceptable carrier or salt. The pharmaceutically acceptable carrier or salt may account for 0.01-99.99 wt %, preferably from 0.1-99.9 wt % of the reagent mentioned above.

In addition, in another aspect of the invention, the invention may provide a free radical-induced intracellular DNA damage reducing medicament or health food which is capable of reducing radical-induced intracellular DNA damage, and the medicament or health food may comprise an effective amount of a Gracilaria sp. extract as an active indigent. Moreover, the invention may further provide a method for preparing a free radical-induced intracellular DNA damage reducing medicament or health food. The method comprises providing an effective amount of a Gracilaria sp. extract, as an active ingredient, in preparation of a free radical-induced intracellular DNA damage reducing medicament or health food as an active indigent.

In one embodiment, the free radical-induced intracellular DNA damage reducing medicament or health food mentioned above may further have an effect for reducing cell death or cell cycle arrest under free radical stress or oxidative stress.

In one embodiment, the free radical-induced intracellular DNA damage reducing medicament or health food mentioned above may further comprise a pharmaceutically acceptable carrier or salt. The pharmaceutically acceptable carrier or salt may account for 0.01-99.99 wt %, or preferably 0.1-99.9 wt % of the free radical-induced intracellular DNA damage reducing medicament or health food.

The above-mentioned pharmaceutically acceptable carrier may comprise, but is not limited to, a solvent, a dispersion medium, a coating, an antibacterial and antifungal agent, or an isotonic and absorption delaying agent. The reagent or the medicament or health food can be formulated into dosage forms for different administration routes utilizing conventional methods.

The pharmaceutically acceptable salt may comprise, but is not limited to, inorganic cation salts including alkali metal salts such as sodium salt, potassium salt or amine salt, alkaline-earth metal salt such as magnesium salt or calcium salt, the salt containing bivalent or quadrivalent cation such as zinc salt, aluminum salt or zirconium salt. In addition, the pharmaceutically acceptable salt may also comprise organic salt including dicyclohexylamine salt, methyl-D-glucamine, and amino acid salt such as arginine, lysine, histidine, or glutamine.

The reagent or the medicament may be administered orally, parenterally by an inhalation spray or via an implanted reservoir. The parenteral method may comprise subcutaneous, intracutaneous, intravenous, intramuscular, intraarticular, intraarterial, intrasynovial, intrasternal, intrathecal, and intraleaional, as well as infusion techniques.

An oral composition of the medicament or health food can comprise, but is not limited to, tablets, capsules, emulsions and aqueous suspensions, dispersions and solutions.

Furthermore, according to the foregoing, in further another aspect of the invention, the invention may provide a free radical-induced intracellular DNA damage reducing skin care product or cosmetic which is capable of reducing radical-induced intracellular DNA damage, and the skin care product or cosmetic may comprise an effective amount of a Gracilaria sp. extract as an active indigent. Moreover, the invention may further provide a method for preparing a free radical-induced intracellular DNA damage reducing skin care product or cosmetic. The method comprises providing an effective amount of a Gracilaria sp. extract in preparation for a free radical-induced intracellular DNA damage reducing skin care product or cosmetic as an active indigent.

In one embodiment, the free radical-induced intracellular DNA damage reducing skin care product or cosmetic mentioned above may further have an effect for reducing cell death or cell cycle arrest under free radical stress or oxidative stress.

In one embodiment, the free radical-induced intracellular DNA damage reducing skin care product or cosmetic mentioned above may further comprise a cosmetically or pharmaceutically acceptable vehicle.

Furthermore, the cosmetically or pharmaceutically acceptable vehicles for all compositions mentioned above may act as a dilutant, dispersant or carrier for the active ingredient. The cosmetically or pharmaceutically acceptable vehicle may comprise materials commonly employed in skin care products such as water, liquid or solid emollients, silicone oils, emulsifiers, solvents, humectants, thickeners, powders, propellants and the like.

The cosmetically or pharmaceutically acceptable vehicle may account for 0.01-99.99 wt % or preferably 0.1-99.9 wt % of the free radical-induced intracellular DNA damage reducing skin care product or cosmetic, and can, in the absence of other adjuncts, form the balance of the skin care product or cosmetic.

Furthermore, other specific ingredients which benefit skin, such as sunscreens, skin-lightening agents, and skin tanning agents may also be included in the skin care product or cosmetic mentioned above. The vehicle may also further include adjuncts such as antioxidants, perfumes, opacifiers, preservatives, colourants and buffers.

Moreover, in one embodiment, all of the skin care products or cosmetics mentioned may be manufactured in a skin spreading form, including, but not limited to creams, ointments, gels, sprays, lotions, skin tonics, shampoos or mousses, etc. Skin sprays are generally composed of aerosolized copolymers, such as polyvinylpyrrolidone, vinyl acetate and the like, and may also function as a setting lotion. Skin gel preparations are similar to sprays in composition, but are in gel and alcohol free form, and can coat the skin. A skin mousse is foam released under pressure from an aerosolized can. Skin creams may be a hydrophobic or hydrophilic cream, ointment, gel, emollient, spray, lotion, skin tonic, shampoo or mousse, suitably with additional ingredients suitable for use in skin creams of types known in the art, and such further ingredients can include petrolatum, waxes, lanolin, silicone, liposomes, vegetable, mineral oils, plasticizers, fragrances, preservatives, a penetration enhancing agent, a pH adjusting agent or other suitable ingredients for skin creams. Such ingredients can moisturize skin, stabilize active compounds, increase the effectiveness of composition-skin contact, increase local concentration and control the release of the skin care product or cosmetic to the skin.

### EXAMPLE

### Materials and Methods

1. Raw Materials

The seaweed Gracilaria tenuistipitata was collected in spring of 2009 from a culture farm at Kouhu beach, Yunlin County, Taiwan, and delivered to the laboratory at 0° C. In the laboratory, the seaweeds were washed with running tap-water to remove epiphytes, saline matter, impurities and sand. Next, the seaweeds were soaked in distilled water twice, and then lyophilized. The lyophilized sample was pulverized and passed through a 60-mesh sieve. The lyophilized sample was ground to fine powder and stored at −40° C.

2. Extraction and Isolation

The dried samples (50 g) obtained in the foregoing were extracted with 1000 ml of deionized water in a mechanical shaker at room temperature for 24 hours. The extract was filtered with Whatman No. 1 filter paper. The filtrate solution was evaporated to dryness at 40±2° C. in a rotary evaporator (Buchi Laboratoriums-Technik, Switzerland) and then lyophilized. The lyophilized extract from Gracilaria was stored in a sealed container at −40° C. until use.

3. Determination of Total Phenolics, Flavonoid, and Ascorbic Acid of the Extract from Gracilaria

The total phenolic compounds in the extract from Gracilaria mentioned above were determined with the Folin-Ciocalteu reagent according to the method of Singleton and Rossi (Am J Enol Vitic, 1965, 16,144-158) using gallic acid as the standard. The total phenolic content is expressed as a gallic acid equivalent in μg per mg of a dry sample. Ascorbic acid was quantitatively determined according to the 2,6-dichloroindophenol-Na dye method in Jones, E.; Hurghes, R. E. Foliar ascorbic acid in some angiosperms (Phytochemistry, 1983, 22(11), 2493-2499). Results are presented on a dry matter basis (μg ascorbic acid per mg of the dry sample). Flavonoid content was determined by using the colorimetric method as described (Journal of Agricultural Research, 1998, 37, 99-105). The content of the total flavonoid was calculated as quercetin from a calibration curve and expressed as a μg quercetin equivalent in per mg of the dry sample.

4. 1,1-diphenyl-2-picrylhydrazyl (DPPH) Radical Scavenging Activity

The 1,1-diphenyl-2-picrylhydrazyl radical scavenging ability of the extract from Gracilaria was determined according to the method of Blois (Nature, 1958, 26, 1199-1200). Briefly, 1 ml of a 1 mM methanolic solution of 1,1-diphenyl-2-picrylhydrazyl was mixed with a 3 ml solution of each extract (1-4 mg/ml). The mixture was then vortexed vigorously and left for 30 minutes at room temperature in the dark. The absorbance was measured at 517 nm and activity was expressed as a percentage of 1,1-diphenyl-2-picrylhydrazyl scavenging relative to control. Percent scavenging activity was calculated as [(Ac−As)/Ac]×100, wherein: As is the absorbance measured with the extract sample in the assay; and Ac is the absorbance of control (without extract sample). Butylated hydroxyanisole (BHA) and ascorbic acid (Vitamin C) were used as positive controls. Results were presented as the means of the experiments performed in triplicate±standard deviation.

5. Cell Cultures

H1299 (human lung adenocarcinoma) cells were cultured in a complete RPMI-1640 supplemented with 10% FBS, 100 U/ml penicillin, 100 μg/ml streptomycin and 0.03% glutamine. The cells were kept at 37° C. in a humidified atmosphere containing 5% CO₂.

6. Plasmid DNA Cleavage Assay

Conversion of the supercoiled (S) form of plasmid DNA to the open-circular (OC) and/or further linear (L) forms was accessed as an indicator of the DNA strand breaks. The reaction mixtures (10 μl) containing 150 ng of plasmid DNA, 0.1 mM FeSO₄, 0.05% H₂O₂ and the above-mentioned extract from Gracilaria at indicated concentrations were incubated at 37° C. for 30 minutes. Subsequently, the reaction was stopped by adding 2 μl of 6× gel loading dye (0.05% bromophenol blue, 40 mM EDTA and 50% glycerol (v/v)) and electrophoresed on a 0.8% agarose gel in a 0.5×TAE buffer at 50 V for 1-2 hours. DNA in the gel was stained with ethidium bromide, visualized and photographed under ultraviolet light. All experiments were repeated three times under identical conditions.

7. Comet-Nuclear Extract (Comet-NE) Assay

Due to use of nuclear extracts (NEs), the comet-NE is more sensitive than the traditional comet assay (Mutat Res, 2007, 629, 133-139; Anal Biochem, 2005, 337, 70-75). The protocol for the comet-NE assay for nuclear extracts was performed as described (J Cell Biochem, 2008, 103, 528-537; DNA Repair (Amst), 2008, 7, 751-761; DNA and Cell Biology, 2009, 28(10), 501-506). In the presence of the above-mentioned extract from Gracilaria at indicated concentrations (0, 0.5, 1, 2 or 4 mg/ml), the H1299 cells were treated with or without H₂O₂ at 37° C. for 30 minutes. The 100 μl aliquots of the suspension containing the H1299 cells (1×10⁶ cells/ml in PBS) were mixed with equal volumes of 1.2% low-melting agarose (in PBS, pH 7.4) and immediately pipetted onto glass slides precoated with 1% regular agarose (in distilled water), respectively. The slides were then immersed in a freshly made ice-cold cell lysis solution (2.5 M NaCl, 100 mM EDTA, 10 mM Tris pH 10, 1% N-laurylsarcosine, 1% Triton X-100 and 10% Dimethylsulfoxide or DMSO), incubated at 4° C. for 2 hours, and then rinsed with deionized water three times. A 20 μl excision mixture containing 0.6 μg NE, 50 mM Hepes-KOH (pH 7.9), 70 mM KCl, 5 mM MgCl₂, 0.4 mM EDTA, 2 mM ATP, 40 mM phosphocreatine and 2.5 mM creatine phosphokinase was added to each slide. The cover slips were covered on the slides, and the slides were incubated at 37° C. for 2 hours in a humidified space for NE digestion. The slides were denatured in 0.3 N NaOH, 1 mM EDTA for 20 min followed by being performed electrophoresis at 20 V, 300 mA for 25 minutes. After washing with deionized water, the slides were neutralized in 0.4 M Tris-HCl, pH 7.5 and stained with 40 μl propidium iodide (PI, 50 μg/ml). Under a fluorescence microscope (TE2000-U; Nikon, Tokyo, Japan), the migration of DNA from the nucleus of each cell was measured with a computer program (http://tritekcorp.com) using the parameter of tail moment, which is defined as the product of the tail length and the fraction of total DNA in the tail.

8. Cell Viability Assay

The 3-(4,5-dimethylthiazol-2-yl)-2,5-diphenyl tetrazolium bromide (MTT) assay was performed as described (J Agric Food Chem, 2010, 58, 8798-8805). In brief, 100 μl of a fresh medium containing 0.5 mg/ml 3-(4,5-dimethylthiazol-2-yl)-2,5-diphenyl tetrazolium bromide was added into each well of a 96-well plate and incubated for 2 hours at 37° C. After removing a 3-(4,5-dimethylthiazol-2-yl)-2,5-diphenyl tetrazolium bromide-containing medium, 100 μl of DMSO was added into each well to solubilize the purple formazan precipitates. The plates were gently shaken for 20 min in the dark and read at 595 nm on a microtiter plate reader.

9. Flow Cytometric Determination of Cell Cycle Histogram by Propidium Iodide Staining

The cell cycle histogram was determined as described (Cancer Detect Prev, 2005, 29, 286-293). In brief, cells were treated with or without H₂O₂ in the presence of the above-mentioned extract from Gracilaria at indicated concentrations (0, 0.5, 1, 2 or 4 mg/ml) for 24 hours. After treatment, cells were collected, washed twice with PBS, and fixed in 70% ethanol overnight. The ethanol containing cells were then centrifuged at 700 rpm for 5 minutes at 4° C. to obtain cell precipitates. Then the cells were resuspended in the PBS buffer containing 10 μg/ml PI (Sigma, St Louis, Mo.) and 10 μg/ml RNase A. After 15 minutes of incubation at room temperature in the dark, the cells were analyzed with a FACScan flow cytometer (Becton-Dickinson, Mansfield, Mass.) and Cell-Quest and Modfit software (Becton-Dickinson, Mansfield, Mass.).

10. Statistical Analysis

All data were presented as mean±SD and the Student's t-test was used to test the mean difference between the two groups.

Results

1. Polyphenols, Flavonoids, and Ascorbic Acid Contents of the Extract from Gracilaria and its 1,1-diphenyl-2-picrylhydrazyl Radical Scavenging Activity

Previous studies indicate that certain polyphenols such as phenolic acid, flavonoid, and tannins (Algae, 2003, 18, 341-347.) and ascorbic acid (Hydrobiologia, 1987, 151/152, 477-481) are potential antioxidant compounds in seaweeds. In present invention, the amount of total polyphenol, flavonoid, and ascorbic were determined to be 98.94±2.43 μg gallic acid equivalent, 22.59±1.08 μg quercetin equivalent, and 1.59±0.18 μg ascorbic acid per mg dry extract from Gracilaria of the invention, respectively. No carotenoid in the extract was detected, probably because the product was an aqueous extract.

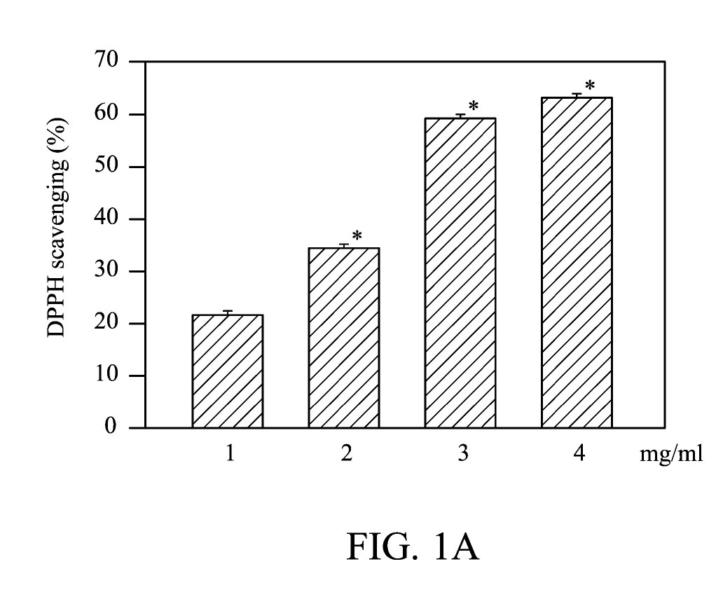

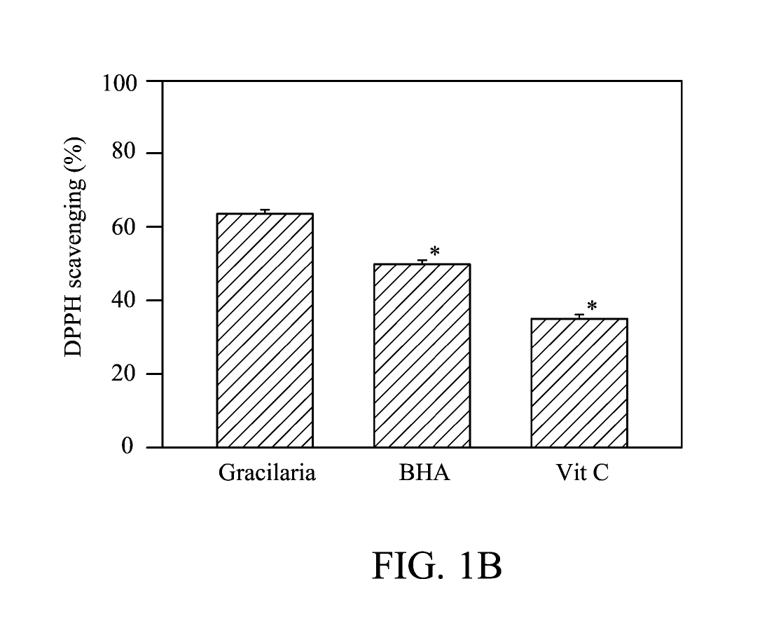

The 1,1-diphenyl-2-picrylhydrazyl radical scavenging is largely applied to assess the free radical scavenging effect of specific compounds or extracts and can be used to evaluate their antioxidant activity in a short time (Food Chemistry, 2008, 107, 40-43; Food Chemistry, 2008, 110,128-36.). The extract from Gracilaria showed concentration-dependant 1,1-diphenyl-2-picrylhydrazyl scavenging activity (FIG. 1A). Moreover, the 1,1-diphenyl-2-picrylhydrazyl scavenging of the 3 and 4 mg/ml extract from Gracilaria reached over 60% (FIG. 1A). In addition, the 1,1-diphenyl-2-picrylhydrazyl scavenging of the 4 mg/ml extract from Gracilaria (60%) was significantly higher than those of the 10 ppm BHA and 100 ppm ascorbic acid (FIG. 1B).

2. The Extract from Gracilaria Prevented Free Radical-Induced DNA Strand Breaks in Plasmid

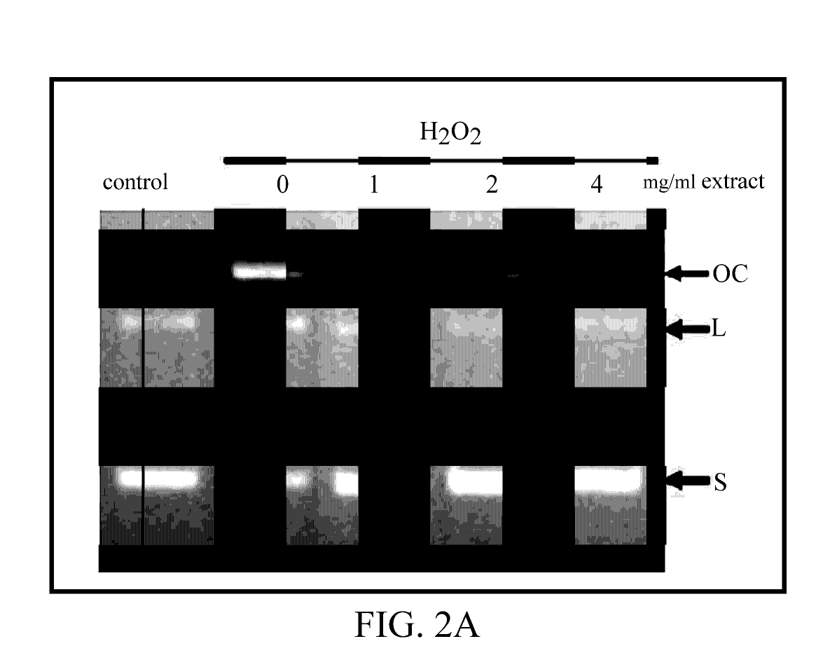

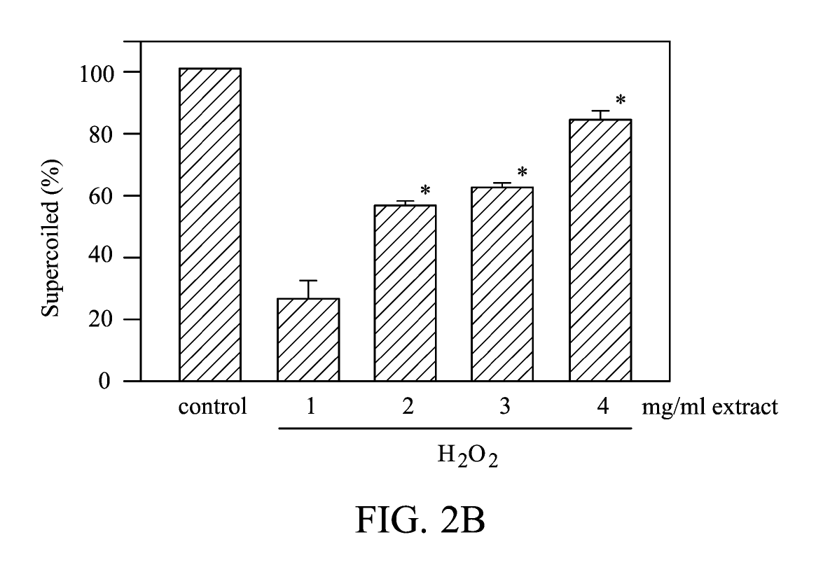

Oxidative DNA damage can be caused by reactive oxygen species (ROS) generated by cellular metabolism or environmental stress, and is believed to be a contributing factor of aging, carcinogenesis and other diseases (FASEB J, 2003, 17, 1195-1214). The conversion of the supercoiled form of plasmid DNA into either an open-circular or linear form has been typically used as an indication of DNA damage (Biochim Biophys Acta, 1994, 1225, 259-263). In the example, the protection effect of the extract from Gracilaria against the pBR322DNA cleavage induced by the free radical stress from the H₂O₂ treatment was accessed. In the absence of H₂O₂, the pBR322 plasmid DNA was mainly in the supercoiled form (S) (FIG. 2A, control). The supercoiled form decreased and was converted to the relaxing open-circular (OC) or linear forms (L) after the addition of FeSO₄ and H₂O₂ (FIG. 2A lane 2). However, in the presence of 1 to 4 mg/ml of the extract from Gracilaria, increasing amounts of pBR322 retained in the supercoiled form (S) (FIG. 2A, lanes 3-5). The relative percentages of the supercoiled pBR322 were reduced to 26.7±4.9 by the H₂O₂ treatment and recovered to 56.2±1.4, 61.8±1.8, 83.5±2.8 in the presence of 1, 2, 4 mg/ml of the extract from Gracilaria, respectively (mean±SD, FIG. 1B). These results indicate that the extract from Gracilaria, was able to neutralize the free radical stress caused by H₂O₂, therefore increasing the stability of DNA molecules.

According to the foregoing, it is shown that the extract from Gracilaria is capable of reducing free radical-induced DNA damage.

3. The Extract from Gracilaria Protected Cellular DNA Damage from Free Radical Stress

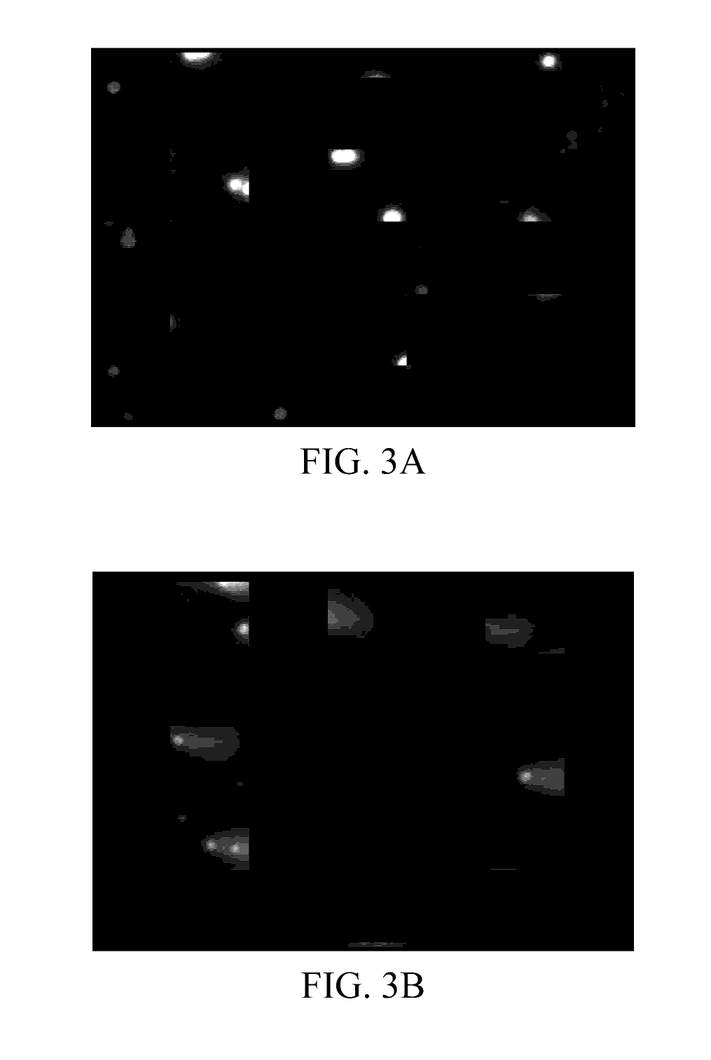

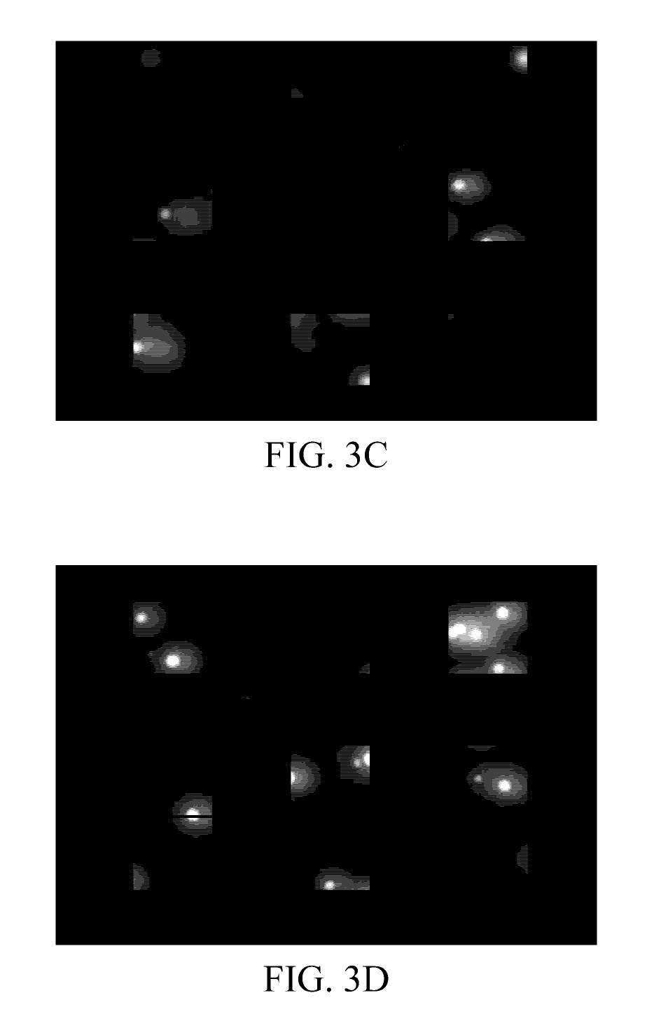

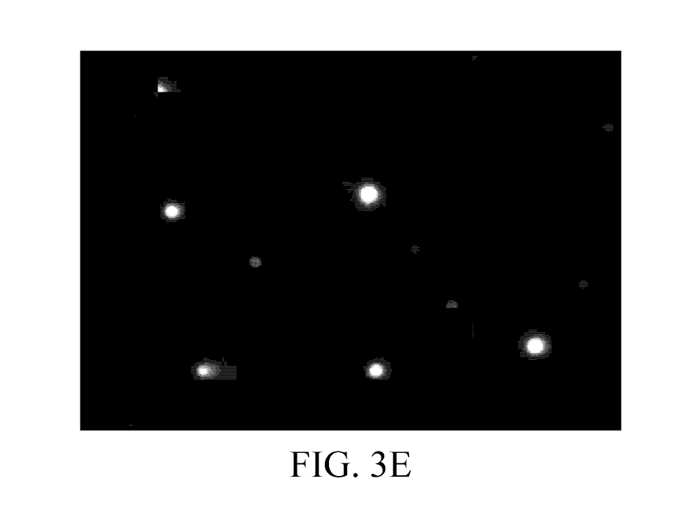

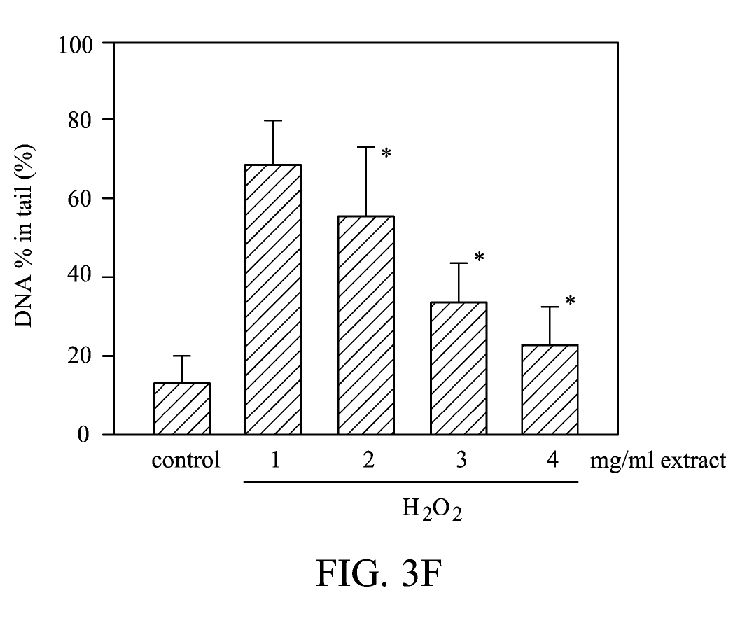

The protective ability of the extract from Gracilaria against oxidative DNA damage was verified in H1299 cells by using comet-NE assay. There were a few “tails” in the untreated control (FIG. 3A). While the DNA trailing was evident with the H₂O₂ treatment (FIG. 3B, H₂O₂ only), it was reduced with the extract from the Gracilaria co-treatments (FIGS. 3C-3E). The average tail moment values (mean±SD) for the NE buffer control, H₂O₂ treatment only, and the extract from Gracilaria treatments (1, 2, and 4 mg/ml) were 12.8±7.3, 69.6±12.0, 53.6±17.9, 32.4±10.5, and 22.1±9.8, respectively (FIG. 3F, P>0.05 between control and 4 mg/ml the extract from Gracilaria treated cells). The extract from Gracilaria showed significant protective effect against free radical-induced DNA damage. These results indicate that the DNA protection effect of the extract from Gracilaria was effective in both a cell-free condition and a cellular condition.

Furthermore, according to the foregoing, it was shown that the extract from Gracilaria is capable of reducing free radical-induced intracellular DNA damage.

4. The Extract from Gracilaria Promoted Cell Survival Under Free Radical Stress

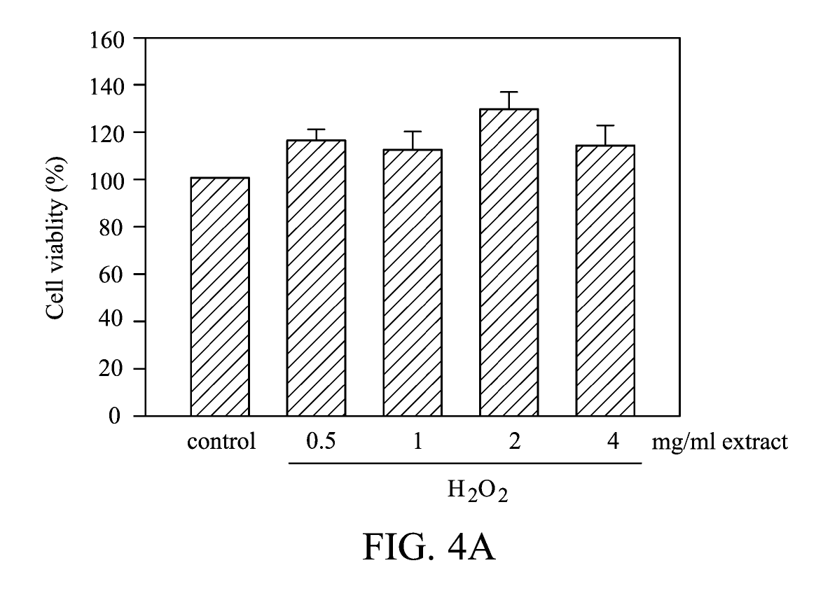

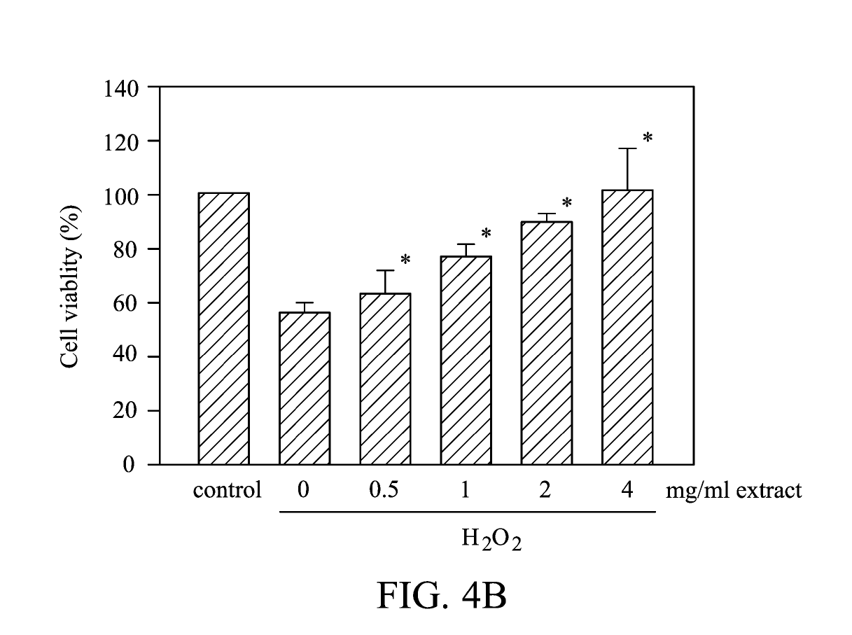

In the example, whether the protective effect of the extract from Gracilaria also promoted the survival of H1299 cells under free radical stress was further tested. H1299 cells were treated with or without H₂O₂ in the presence of 0.5, 1, 2, and 4 mg/ml of extract from Gracilaria for 24 hours. The cell viability was determined by a 3-(4, 5-dimethylthiazol-2-yl)-2,5-diphenyl tetrazolium bromide (MTT) assay and normalized to that of untreated H1299 cells. Treating H1299 with the extract from Gracilaria with up to 4 mg/ml alone did not have adverse effects on the cell viability (FIG. 4A). In the presence of H₂O₂-induced free radical stress, the extract from Gracilaria increased cell viability in a dose-dependent manner, increasing the cell viability of H₂O₂-treated cells from 55.6±3.7 to 63.3±8.3, 76.4±4.6, 89.0±3.2 and 100.3±16.0 with 0.5, 1, 2 and 4 mg/ml the extract from Gracilaria, respectively (FIG. 4B). Notably, the viability of the H1299 cells treated with both H₂O₂ and 4 mg/ml of the extract from Gracilaria were not significantly different from the control cells (p>0.05, FIG. 4, lane 6).

5. The Extract from Gracilaria Relieved the Cell Cycle Arrest Caused by Free Radical Stress

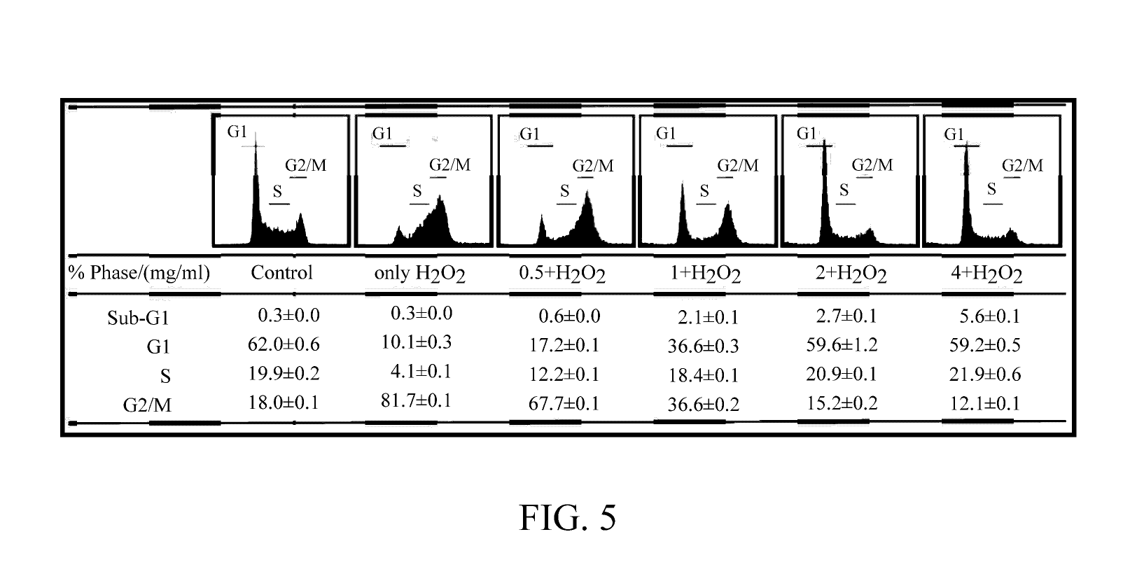

The cell cycle distributions of the H1299 cells treated with H₂O₂ in the presence of 0-4 mg/ml of the extract from Gracilaria for 24 hours were further analyzed by flow cytometry. The control H1299 cells displayed a major G1 peak and a minor G2/M peak comprising 62.0% and 18.0% of the cell population, respectively (FIG. 5, panel 1). The H₂O₂-treated H1299 cells showed prominent G2/M population of 81.7%, which was a clear sign of cell cycle arrest caused by DNA damage (FIG. 5, panel 2). However, the G2/M arrest was increasingly relieved by co-incubation with the extract from Gracilaria and virtually disappeared in the presence of 2-4 mg/ml of the extract from Gracilaria (FIG. 5, panels 3-6). This result indicates that the extract from Gracilaria is able to prevent DNA damage and ensure the normal progression of cell cycles.

### DISCUSSION

In the invention, the anti-free radical activity, especially the cellular protective effect against free radical-induced DNA damage of the extract from Gracilaria was investigated. Note that ROS induces the formation of 8-oxoguanine, accumulation of which could induce base pairing mismatch, miscoding of proteins, DNA mutation and even instability of the genome (Circ Res, 2001, 88, 733-739.). The extract from Gracilaria effectively suppressing ROS-induced DNA strand break in the cell-free assay was confirmed in the invention. Further examination using the sensitive comet-NE assay to measure cellular DNA damage and cell cycle profiling also indicated that the extract from Gracilaria protected the H1299 cells from free radical stress and therefore increased the survival of cells under free radical stress.

A previous report indicated that the boiling extract of G. tenuistipitata exhibits radical scavenging activities with an IC₅₀ of 103 mg/ml in 1,1-diphenyl-2-picrylhydrazyl radical scavenging assay (Plant Foods Hum Nutr, 2009, 64, 218-223). In the invention, it was shown that the room temperature extract from Gracilaria of the invention exhibits more potent antioxidant activity, displaying 60% 1,1-diphenyl-2-picrylhydrazyl scavenging activity at merely 3 mg/ml.

Considering the fast growing speed of seaweed and the extensive use of seaweed extracts in food and cosmetic industries, the room temperature extract from Gracilaria of the invention therefore represent an attractive multifunctional alternative in the applications in food and cosmetic products.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A free radical-induced intracellular DNA damage reducing reagent, comprising:
an effective amount of a Gracilaria sp. extract, serving as an active ingredient,
wherein the free radical-induced intracellular DNA damage reducing reagent is capable of reducing free radical-induced intracellular DNA damage.

2. The free radical-induced intracellular DNA damage reducing reagent as claimed in claim 1, wherein the Gracilaria sp. comprises Gracilaria tenuistipitata, Gracilaria coforvoides, Gracilaria gigas, Gracilaria chorda, Gracilaria lichenoides or Gracilaria compressa.

3. The free radical-induced intracellular DNA damage reducing reagent as claimed in claim 1, wherein a process for obtaining the Gracilaria sp. extract comprises performing a first extraction step to the Gracilaria sp. to obtain a first extract, and the first extraction step comprises extracting the Gracilaria sp. with a solvent.

4. The free radical-induced intracellular DNA damage reducing reagent as claimed in claim 3, wherein the first extraction step is performed at room temperature.

5. The free radical-induced intracellular DNA damage reducing reagent as claimed in claim 3, wherein the solvent comprises water, methanol, ethanol, propanol or the combination thereof.

6. The free radical-induced intracellular DNA damage reducing reagent as claimed in claim 3, wherein a time for the first extraction step is about 15-30 hours.

7. The free radical-induced intracellular DNA damage reducing reagent as claimed in claim 3, wherein the process for obtaining the Gracilaria sp. extract further comprises filtering the first extract to obtain a filtrate and a residue, and wherein the filtrate is a second extract.

8. The free radical-induced intracellular DNA damage reducing reagent as claimed in claim 7, wherein the process for obtaining the Gracilaria sp. extract further comprises:
performing a second extraction step to the residue to obtain an extract of the residue, wherein the second extraction step comprises extracting the residue with a second solvent at room temperature;
filtering the extract of the residue to obtain a second filtrate; and
mixing the second filtrate with the second extract to obtain a third extract.

9. The free radical-induced intracellular DNA damage reducing reagent as claimed in claim 8, wherein a time for the second extraction step is about 15-30 hours.

10. The free radical-induced intracellular DNA damage reducing reagent as claimed in claim 3, wherein the solvent is water and the Gracilaria sp. is Gracilaria tenuistipitata.

11. The free radical-induced intracellular DNA damage reducing reagent as claimed in claim 1, wherein the free radical-induced intracellular DNA damage comprises free radical-induced intranuclear DNA damage.

12. A free radical-induced intracellular DNA damage reducing medicament or health food, comprising:
an effective amount of a Gracilaria sp. extract, serving as an active ingredient,
wherein the free radical-induced intracellular DNA damage reducing medicament or health food is capable of reducing free radical-induced intracellular DNA damage.

13. The free radical-induced intracellular DNA damage reducing medicament or health food as claimed in claim 12, wherein the Gracilaria sp. comprises Gracilaria tenuistipitata, Gracilaria coforvoides, Gracilaria gigas, Gracilaria chorda, Gracilaria lichenoides or Gracilaria compressa.

14. The free radical-induced intracellular DNA damage reducing medicament or health food as claimed in claim 12, wherein a process for obtaining the Gracilaria sp. extract comprises performing a first extraction step to the Gracilaria sp. to obtain a first extract, and the first extraction step comprises extracting the Gracilaria sp. with a solvent.

15. The free radical-induced intracellular DNA damage reducing medicament or health food as claimed in claim 14, wherein the solvent is water and the Gracilaria sp. is Gracilaria tenuistipitata.

16. The free radical-induced intracellular DNA damage reducing medicament or health food as claimed in claim 12, wherein the free radical-induced intracellular DNA damage comprises free radical-induced intranuclear DNA damage.

17. The free radical-induced intracellular DNA damage reducing medicament or health food as claimed in claim 12, further comprising a pharmaceutically acceptable carrier or salt.

18. A free radical-induced intracellular DNA damage reducing skin care product or cosmetic, comprising:
an effective amount of a Gracilaria sp. extract, serving as an active ingredient,
wherein the free radical-induced intracellular DNA damage reducing skin care product or cosmetic is capable of reducing free radical-induced intracellular DNA damage.

19. The free radical-induced intracellular DNA damage reducing skin care product or cosmetic as claimed in claim 18, wherein the Gracilaria sp. comprises Gracilaria tenuistipitata, Gracilaria coforvoides, Gracilaria gigas, Gracilaria chorda, Gracilaria lichenoides or Gracilaria compressa.

20. The free radical-induced intracellular DNA damage reducing skin care product or cosmetic as claimed in claim 18, wherein a process for obtaining the Gracilaria sp. extract comprises performing a first extraction step to the Gracilaria sp. to obtain a first extract, and the first extraction step comprises extracting the Gracilaria sp. with a solvent.

21. The free radical-induced intracellular DNA damage reducing skin care product or cosmetic as claimed in claim 20, wherein the solvent is water and the Gracilaria sp. is Gracilaria tenuistipitata.

22. The free radical-induced intracellular DNA damage reducing skin care product or cosmetic as claimed in claim 18, wherein the free radical-induced intracellular DNA damage comprises free radical-induced intranuclear DNA damage.

23. The free radical-induced intracellular DNA damage reducing skin care product or cosmetic as claimed in claim 18, further comprising a cosmetically or pharmaceutically acceptable vehicle.

24. A method for preparing a free radical-induced intracellular DNA damage reducing reagent, comprising:
providing an effective amount of a Gracilaria sp. extract, as an active ingredient, in preparation of a free radical-induced intracellular DNA damage reducing reagent.

25. A method for preparing a free radical-induced intracellular DNA damage reducing medicament or health food, comprising:
providing an effective amount of a Gracilaria sp. extract, as an active ingredient, in preparation of a free radical-induced intracellular DNA damage reducing medicament or health food.

26. A method for preparing a free radical-induced intracellular DNA damage reducing skin care product or cosmetic, comprising:
providing an effective amount of a Gracilaria sp. extract, as an active ingredient, in preparation of a free radical-induced intracellular DNA damage reducing skin care product or cosmetic.
